(19) United States
(12) Patent Application Publication
(10) Pub. No.: US 2005/0042172 A1
(43) Pub. Date: Feb. 24, 2005

(54) Administration of medicaments by vaporisation

(76) Inventor: Brian Anthony Whittle, Hornesa, Yorkshire (GB)

Correspondence Address: WOLF GREENFIELD & SACKS, PC;FEDERAL RESERVE PLAZA, 600 ATLANTIC AVENUE, BOSTON, MA, 02210-2211, US

(21) Appl. No.: 10/493,757

(22) PCT Filed: Oct. 31, 2002
(86) PCT No.: PCT/GB02/04984

(30) Foreign Application Priority Data
Oct. 31, 2001 (GB) 0126151.0

(51) Int. Cl. A61L 9/04; A61K 9/14; B29B 9/00

(52) U.S. Cl. 424/46; 264/5

(57) ABSTRACT

A method of making a medicament which is a vapour comprising heating a composition to a temperature not exceeding 500° C. for a time of less than 10 seconds. The composition is non-volatile at 75° C. and generates a vapour free of pyrolysis products when heated in this way. The vapour is produced in a portion of air smaller than the mean respiratory tidal volume. A composition suitable for using in such a method is also disclosed.

Administration of medicaments via the respiratory tract is one of the fastest ways of producing a therapeutic effect. This route is widely used for the administration of anaesthetics, bronchodilator (β-adrenergic agonists and parasympathetic antagonist) drugs, anti-asthmatic, analgesic, and smooth muscle relaxant drugs. The applicant has investigated the range of compounds that can be so delivered, and has devised improvements to compositions and methods of administration via the respiratory tract.

Drugs are administered into the respiratory tract in a number of ways. General anaesthetics in the form of gases are given into the respiratory tract. Other agents such as glyceryl trinitrate and other nitrates and nicotine which are volatile at ambient room temperature can also be given by inhalation. Administration via the respiratory tract is also employed for a number of other agents such as corticosteroids and sympathomimetics for the treatment of asthma. The advantages of this route of administration are that:
(i) drugs that have a local action on the lining of the lungs have immediate access to the mucus membrane of the lung. The barrier separating air from the systemic circulation in the terminal bronchioles is one to several cells thick and provides a rapid means of absorption of drugs into the systemic circulation;
(ii) where the medicament is given in the form of a suspension or mist (aerosol) it is possible to control the area of the lung that is reached;
(iii) lungs have a good blood supply and absorption into the systemic circulation is rapid;
(iv) medication absorbed via the lungs is quickly distributed by the heart and arterial system to major organs such as the brain and skin without first passing through the liver. Drugs that are absorbed from the gastrointestinal tract on the other hand, are taken into the hepatic portal system are delivered first into the liver. In the liver, a large proportion of the dose may be destroyed by this so-called “first pass effect”.

There are many devices designed to produce either a fine particle suspension or aerosol for administration of medications, which are not soluble or volatile, into the respiratory tract.

Dry particle inhalers (DPIs) deliver a cloud of particles into the flow of air breathed in. To be effective the particles need to be static-free, and within a narrow range of size. The process of size reduction may cause the particles to become electrostatically charged and agglomerate together to larger particles.

Particles with mean dimensions greater that 15 microns have sufficient mass to hit the back of the throat when delivered from a conventional aerosol dispenser. They therefore may miss being taken into the respiratory tract and are swallowed. The swallowed drug may still be effective although the pharmacokinetics of its absorption and ultimate fate are different from material which is absorbed form the respiratory tract. Particles of mean size of 5-15 microns tend to be deposited high up in the bronchial tree where absorption is less efficient that from the terminal bronchioles. Between 2 and 5 microns the particles reach the level of the bronchi and terminal bronchioles and exert their effect. Below approximately 1 micron mean diameter Brownian movement is exhibited by the particles and a proportion of particles of this size tend to be breathed out in the expired air.

Metered dose inhalers (MDIs) are aerosol presentations which typically deliver 5-200 μl of a solution of the drug that is broken up into a fine spray. The restriction on size of particle is roughly the same as for DPIs.

However, the requirements on optimum sizes of particles both for DPIs and MDIs are illustrative, and considerable variation is experienced. A factor which is important is that when delivered from a nozzle, under pressure generated by a propellant, the particle may change in size, particularly as it travels down the respiratory tract where the inspired air has a high relative humidity.

The compositions and devices used to deliver medicaments into the respiratory tract are used at ambient room temperature. Surprisingly, it has been found that compositions which are solids or gels at ambient room temperature can be converted into a vapour under controlled conditions so that the vapour, when admixed with inspired air, can be used to deliver medicament to all parts of the respiratory tract.

The applicant hypothesised that some of the disadvantages of currently available inhalation devices could be overcome by producing a quantity of vapour from a portion of a composition at the time of use, and diluting the vapour with inspired air.

Therefore, in a first aspect the invention provides a method of making a medicament which is a vapour comprising or consisting of at least one therapeutic substance or a precursor thereof, which method comprises heating a composition to a temperature not exceeding 500° C. for a time of less than 10 seconds and thereby generating a vapour comprising or consisting of at least one therapeutic substance or a precursor thereof, wherein the composition is non-volatile at 25° C. but is capable of generating a vapour comprising at least one therapeutic substance or a precursor thereof which is substantially free of any products of pyrolysis when heated to a temperature not exceeding 500° C. for a time not exceeding 10 seconds.

In a second aspect the invention provides a method of administering a vapour or its condensate comprising or consisting of at least one therapeutic substance or a precursor thereof by inhalation, which method comprises heating a composition to a temperature not exceeding 500° C. for a time not exceeding 10 seconds to generate a vapour comprising or consisting of at least one therapeutic substance or a precursor thereof in a portion of air smaller than the mean respiratory tidal volume, and inhaling the vapour so-produced or its condensate in admixture with inspired air, wherein the composition is non-volatile at 25° C. but is capable of generating a vapour comprising at least one therapeutic substance or a precursor thereof which is substantially free of any products of pyrolysis when heated to a temperature not exceeding 500° C. for a time not exceeding 10 seconds.

This method may be used to administer a vapour or its condensate to a human or animal subject. In this context the term “mean respiratory tidal volume” refers to the mean respiratory tidal volume of the subject to which the vapour, or its condensate, is administered. In humans tidal volume will vary depending on the age, sex and health of the subject. A typical tidal volume for a healthy adult male is in the region of 500 ml. For any given subject, tidal volume may be easily measured using techniques well known in the art (e.g. using a spirometer).

In a third aspect the invention provides a composition formulated for administration of a vapour or its condensate, which vapour comprises or consists of at least one therapeutic substance or a precursor thereof, wherein the composition is non-volatile at 25° C. but is capable of generating a vapour comprising at least one therapeutic substance or a precursor thereof which is substantially free of any products of pyrolysis when heated to a temperature not exceeding 500° C. for a time not exceeding 10 seconds.

The feasibility of administering an active substance by inhalation of a vapour can be demonstrated by the act of cigarette smoking. Cigarette tobacco contains up to 8% of nicotine is liberated and volatilised during smoking. Some nicotine will be destroyed by the high temperature immediately behind the burning ember. The same holds in the case of marijuana which may be smoked alone or in combination with tobacco. The smoke produced in this way contains the active component but also contains the products of pyrolysis, particularly tars and dust particles. These particles may be deposited high in the respiratory tract and some of the adverse effects of cigarette smoking are due to the deposition of carcinogenic tars at the bifurcation of the branchial tree. Here, an eddy pattern slows down the stream of inhaled particles and causes a locally high concentration of carcinogens and other irritants.

There is no doubt that administration by inhalation is a very effective way of administering some medicaments. Smoking is not, however, an acceptable way of administering medication. At a temperature greater than 425° F. (approximately 218° C.) cellulose and other material in cigarette tobacco and paper are converted into polyaromatic hydrocarbons which are thought to be responsible for the carcinogenic changes in the lung. Temperatures in a burning cigarette ember may be up to 900-1,000° C. for a brief period, and between draws the temperature may still be in excess of 400°. Burning the medication is not an efficient way of generating heat.

Critical examination of the requirements for efficient administration of medicaments via inhalation of a vapour require a device and compositions in which there is:
(i) Vaporisation at a controlled temperature;
(ii) Generation of vapour at the time of use;
(iii) The generated vapour is substantially free of particulate matter;
(iv) Control of the quantity and rate of drug delivery; Limitation of heat so that polyaromatic hydrocarbons (PAHs) are not formed;
(v) A quantum of heat is used in vaporisation such that when mixed with the rest of the inspired air the lining of the respiratory tract is not scorched.

Surprisingly, it has been found that by using an electrically heated resistor, a quantum of heat can be applied to a unit dose of a composition so that it yields its content as vapour into a space with a defined volume. When admixed with inspired air the charge of dilute vapour has a therapeutic effect. This is achieved without taking the time and temperature of vaporisation above that at which PAH formation can occur.

It has been discovered that the formation of undesirable products of pyrolysis is not a step function occurring at a specific temperature but is a function of a time-temperature product. If the period of heating is short, then it is possible to gain higher efficiency of vaporisation by using higher temperatures. The quantum of heat which has been found to produce virtually complete vaporisation of a quantity of 10 mg of an extract of cannabis was 40-60 watt seconds. No PAHs were detected by GLC analysis following vaporisation of cannabis extract at temperatures of 375° C. for a period of 5 seconds.

In preferred embodiments of the method of the invention the composition may be heated to temperatures in the range 100-500° C., more preferably 100-400° C., more preferably 100-300° C., more preferably 150-250° C., depending on the precise nature of the composition. It is essential that the composition is capable of generating a vapour which is substantially free of any products of pyrolysis when heated to the chosen temperature (for the chosen period of time).

The composition is heated for a period of time which is not more than 10 seconds, preferably in the range 0.1-5 seconds, and most preferably about 1 second, depending on the nature of the composition. The composition must be capable of generating a vapour which is substantially free of any products of pyrolysis when heated for this period of time (at the chosen temperature).

Using the method of the invention therapeutic substances may be administered to the respiratory tract as a vapour or its condensate, since it is possible that the vapour comprising or consisting of the therapeutic substance may condense within the respiratory tract.

Compositions for use in the methods of the invention comprise one or more therapeutic substances or precursors thereof and when heated generate a vapour which may also comprise one or more therapeutic substances or precursors thereof. However, it is to be understood that in many instances the “therapeutic substances or precursors thereof” present in the vapour generated by heating of a given composition may differ from the “therapeutic substances or precursors thereof” original present in the composition in terms of chemical structure. In other words, it is often not the case that the vapour generated from a given composition is chemically identical to the original composition. Some of the possibilities are summarised below, however this is intended to be illustrative of rather than limiting to the invention:

The composition may comprise therapeutic substances in pharmacologically active form. On heating, the composition generates a vapour which also comprises the therapeutic substances in pharmacologically active form.

The composition may contain a pharmacologically inactive precursor of a therapeutic substance. On heating, the precursor is converted into the corresponding therapeutic substance by the action of heat during vaporisation, thus giving a vapour which comprises the pharmacologically active therapeutic substance.

The composition may contain a pharmacologically inactive precursor of a therapeutic substance. On heating, the composition generates a vapour which also comprises the precursor in pharmacologically inactive form. The precursor is converted into the pharmacologically active therapeutic substance in situ in the respiratory tract.

Each of the above examples results in the delivery of a pharmacologically active therapeutic substance to the respiratory tract.

The term “therapeutic substance” encompasses essentially any substance which it is desired to administer to a human or animal subject for the purpose of providing some therapeutic benefit to the subject. “Therapeutic benefit” in this context includes prophylactic treatment for the purposes of preventing disease, as well as treatment aimed at alleviating the symptoms of disease. Suitable “therapeutic substances” include conventional pharmacologically active pharmaceutical substances and medicaments and also extracts from plants which are known to have therapeutic activity.

The term “precursor of a therapeutic substance” refers to a substance which is pharmacologically inactive but is capable of being converted into a pharmacologically active therapeutic substance. In particular, the term “precursor of a therapeutic substance” encompasses substances which are present in a pharmacologically inactive form in the composition but are converted into a pharmacologically active form by the application of heat during the vaporisation process, thus giving the corresponding “therapeutic substance” in the resultant vapour. Specific examples include the acid forms of cannabinoids which may be converted to the active free cannabinoid form by the application of heat during vaporisation. Compositions comprising cannabinoid acids as precursors of therapeutic substances thus generate vapours containing the corresponding free cannabinoids which are therepeutic substances.

The term “precursors of therapeutic substances” is also used herein to refer to substances which are present in a pharmacologically inactive form in the vapour generated by heating of a composition but are converted into a pharmacologically active form in situ when introduced into the respiratory tract.

The therapeutic substance(s), or precursor(s) thereof, generated by heating of the composition preferably have a boiling point or produce substantial vapour pressure in the range 75° C.-500° C., more preferably in the range 180° C.-375° C. The term “substantial vapour pressure” is defined as meaning that the substance generates an effective amount of vapour (preferably an amount of vapour which is sufficient to be of therapeutic benefit when administered to a patient in admixture with inspired air) at the stated temperature.

In a preferred embodiment the therapeutic substance included in the composition is at least one cannabis extract.

In a most preferred embodiment of the methods of the invention the composition consists of at least one cannabis extract.

In the context of this application the terms “cannabis extract” or “extract from a cannabis plant”, which are used interchangeably encompass “Botanical Drug Substances” derived from cannabis plant material. A Botanical Drug Substance is defined in the Guidance for Industry Botanical Drug Products Draft Guidance, August 2000, US Department of Health and Human Services, Food and Drug Administration Centre for Drug Evaluation and Research as: “A drug substance derived from one or more plants, algae, or macroscopic fungi. It is prepared from botanical raw materials by one or more of the following processes: pulverisation, decoction, expression, aqueous extraction, ethanolic extraction, or other similar processes.” A botanical drug substance does not include a highly purified or chemically modified substance derived from natural sources. Thus, in the case of cannabis, “botanical drug substances” derived from cannabis plants do not include highly purified, Pharmacopoeial grade cannabinoids.

A “plant extract” is an extract from a plant material as defined in the Guidance for Industry Botanical Drug Products Draft Guidance, August 2000, US Department of Health and Human Services, Food and Drug Administration Centre for Drug Evaluation and Research.

“Plant material” is defined as a plant or plant part (e.g. bark, wood, leaves, stems, roots, flowers, fruits, seeds, berries or parts thereof) as well as exudates.

The term “Cannabis plant(s)” encompasses wild type Cannabis sativa and also variants thereof, including cannabis chemovars which naturally contain different amounts of the individual cannabinoids, Cannabis sativa subspecies indica including the variants var. indica and var.kafiristanica, Cannabis indica and also plants which are the result of genetic crosses, self-crosses or hybrids thereof. The term “Cannabis plant material” is to be interpreted accordingly as encompassing plant material derived from one or more cannabis plants. For the avoidance of doubt it is hereby stated that “cannabis plant material” includes dried cannabis biomass.

“Botanical drug substances” derived from cannabis plants include primary extracts prepared by such processes as, for example, maceration, percolation, extraction with solvents such as C1 to C5 alcohols (ethanol), Norflurane (HFA134a), HFA227 and liquid carbon dioxide under pressure. The primary extract may be further purified for example by supercritical or subcritical extraction, vaporisation and chromatography. When solvents such as those listed above are used, the resultant extract contains non-specific lipid-soluble material. This can be removed by a variety of processes including “winterisation”, which involves chilling to −20° C. followed by filtration to remove waxy ballast, extraction with liquid carbon dioxide and by distillation.

Preferred “cannabis extracts” include those which are obtainable by using any of the methods or processes specifically disclosed herein for preparing extracts from cannabis plant material. The extracts are preferably substantially free of waxes and other non-specific lipid soluble material but preferably contain substantially all of the cannabinoids naturally present in the plant, most preferably in substantially the same ratios in which they occur in the intact cannabis plant. In a preferred embodiment, substantially all the cannabinoids present in the extract will be in the same chemical form in which they occur in the cannabis plant, this being predominantly the cannabinoid acid form.

Botanical drug substances are formulated into “Botanical Drug Products” which are defined in the Guidance for Industry Botanical Drug Products Draft Guidance, August 2000, US Department of Health and Human Services, Food and Drug Administration Centre for Drug Evaluation and Research as: “A botanical product that is intended for use as a drug; a drug product that is prepared from a botanical drug substance.”

In a further preferred embodiment the therapeutic substance included in the composition may comprise one or more natural or synthetic cannabinoids.

In this embodiment the “cannabinoids” may be highly purified, Pharmacopoeial Grade substances and may be obtained by purification from a natural source or via synthetic means. The cannabinoids will include, but are not limited to, tetrahydrocannabinoids, their precursors, alkyl (particularly propyl) analogues, cannabidiols, their precursors, alkyl (particularly propyl) analogues, and cannabinol.

In a preferred embodiment the therapeutic substance may comprise tetrahydrocannabinol, Δ⁹-tetrahydrocannabinol, Δ⁹-tetrahydrocannabinol propyl analogue, cannabidiol, cannabidiol propyl analogue, cannabinol, cannabichromene, cannabichromene propyl analogue, cannabigerol or any mixture thereof.

The compositions may comprise specific ratios of the cannabinoids cannabidiol (CBD) to tetrahydrocannabinol (THC). As discussed in the applicant's co-pending application (GB 0121715.7), specific combinations of these cannabinoids have been found to be clinically useful in the treatment or management of specific diseases or medical conditions.

The compositions according to the invention may comprise extracts of the cannabis plant and also individual cannabinoids, or synthetic analogues thereof, whether or not derived from cannabis plants, and also combinations of cannabinoids. “Cannabis plants” includes wild type Cannabis sativa and variants thereof, including cannabis chemovars which naturally contain different amounts of the individual cannabinoids. In particular, the compositions may include cannabis based medicine extracts (CBME).

Although preferred therapeutic substances include cannabis extracts and cannabinoids, the utility of the invention is by no means limited to administration of these substances. A list of therapeutic substances which may be included in the compositions of the invention and administered in the form of a vapour according to the method of the invention is given in table 1.

TABLE 1
Therapeutic substances which can be
administered in the form of a vapour.

| CLASS OF MEDICAMENT | EXAMPLE OF MEDICAMENT |
|---|---|
| Alkaloid-rich extracts of | Hyoscine |
| Belladonna atropa | Hyoscamine |
| | Atropine |
| Alkaloid-rich extracts of | Gallanthamine |
| Gallanthus spp. | |
| Alkaloid-rich extracts of | Gallanthamine |
| Narcissus spp. | |
| Alkaloid-rich extracts | Morphine |
| of opium | Codeine |
| | Diamorphine |
| Alkaloid-rich extracts of | Pilocarpine salicylate |
| Pilocarpine | |
| Anti-asthmatics | Terbutaline |
| Antibacterials | Chlorocresol |
| Anti-emetics | Ondansetron |
| | Prochlorperazine |
| Antifungals | Fluconazole |
| Anti-inflammatory agents | Benzidamine |
| | Pyroxicam |
| Antivirals | Acyclovir |
| | Zidovudine |
| Steroid | Beclomethasone |
| Cannabinoid-rich fractions of | |
| Cannabis | |
| sativa and Cannabis indica, | |
| and chemovars derived from | |
| them | |
| Cannabinoids | Δ⁻⁹ Tetrahydrocannabinol |
| | (THC) |
| | Cannabidiol (CBD) |
| | Cannabivarins (THCV) |
| | Cannabinol (CBN) |
| Cannabinoid-rich fractions | THCA |
| containing cannabinoids other | CBDA |
| than THC, CBD or CBN as the | |
| most abundant component | |
| Cardiovascular Agents | Nifedipine |
| | Diltiazem |
| | Verapamil |
| Centrally acting analgesics | Butorphenol |
| | Buprenorphine |
| | Fentanyl |
| Steroid ester | Fluticasone propionate |
| Sympathomemetic amines | Salbutamol |

The compositions described herein are suitable for use and intended for use in methods of treatment of the human or animal body by therapy. In particular, the compositions may be heated to produce a vapour, which vapour (or its condensate) is then administered to the respiratory tract by inhalation.

In preferred embodiments, the invention provides compositions comprising cannabis extracts, natural or synthetic cannabinoids or mixtures thereof which can be administered in the form of a vapour for the treatment of pain, particularly pain unresponsive to opioid analgesics, pain arising from neuropathic and neurogenic conditions, dysmenorrhoea, inflammatory pain, particularly that associated with rheumatoid arthritis, depression, migraine, asthma, epilepsy, post-operative pain, glaucoma, chemotherapy-induced nausea and vomiting, relief of pain and muscle spasm in multiple sclerosis, and loss of appetite and anorexia, particularly in AIDS patients.

Cannabis has been used medicinally for many years, and in Victorian times was a widely used component of prescription medicines. It was used as a hypnotic sedative for the treatment of “hysteria, delirium, epilepsy, nervous insomnia, migraine, pain and dysmenorrhoea”. The use of cannabis continued until the middle of the twentieth century, and its usefulness as a prescription medicine is now being re-evaluated. The discovery of specific cannabinoid receptors and new methods of administration have made it possible to extend the use of cannabis-based medicines to historic and novel indications.

The recreational use of cannabis prompted legislation which resulted in the prohibition of its use. Historically, cannabis was regarded by many physicians as unique; having the ability to counteract pain resistant to opioid analgesics, in conditions such as spinal cord injury, and other forms of neuropathic pain including pain and spasm in multiple sclerosis.

In the United States and Caribbean, cannabis grown for recreational use has been selected so that it contains a high content of tetrahydrocannabinol (THC), at the expense of other cannabinoids. In the Merck Index (1996) other cannabinoids known to occur in cannabis such as cannabidiol and cannabinol were regarded as inactive substances. Although cannabidiol was formerly regarded as an inactive constituent there is emerging evidence that it has pharmacological activity, which is different from that of THC in several respects. The therapeutic effects of cannabis cannot be satisfactorily explained just in terms of one or the other “active” constituents.

It has been shown that tetrahydrocannabinol (THC) alone produces a lower degree of pain relief than the same quantity of THC given as an extract of cannabis. The pharmacological basis underlying this phenomenon has been investigated. In some cases, THC and cannabidiol (CBD) have pharmacological properties of opposite effect in the same preclinical tests, and the same effect in others. For example, in some clinical studies and from anecdotal reports there is a perception that CBD modifies the psychoactive effects of THC. This spectrum of activity of the two cannabinoids may help to explain some of the therapeutic benefits of cannabis grown in different regions of the world. It also points to useful effects arising from combinations of THC and CBD. These have been investigated by the applicant. Table 2 below shows the difference in pharmacological properties of the two cannabinoids.

TABLE 2

| Effect | THC | THCV | CBD | CBDV | Reference |
|---|---|---|---|---|---|
| CB₁ (Brain receptors) | ++ | | ± | | Pertwee et al, |
| | | | | | 1998 |
| CB₂ (Peripheral | + | | − | | |
| receptors) | | | | | |
| CNS Effects | | | | | |
| Anticonvulsant † | −− | | ++ | | Carlini et al, |
| | | | | | 1973 |
| Antimetrazol | − | | − | | GW Data |
| Anti-electroshock | − | | ++ | | GW data |
| Muscle Relaxant | −− | | ++ | | Petro, 1980 |
| Antinociceptive | ++ | | + | | GW data |
| Catalepsy | ++ | | ++ | | GW data |
| Psychoactive | ++ | | − | | GW data |
| Antipsychotic | − | | ++ | | Zuardi et |
| | | | | | al, 1991 |
| Neuroprotective | + | | ++ | | Hampson |
| antioxidant | | | | | A J et al, |
| activity* | ++ | | − | | 1998 |
| Antiemetic | + | | + | | |
| Sedation (reduced | ++ | | | | Zuardi et al, |
| spontaneous activity) | | | | | 1991 |
| Appetite stimulation | | | ++ | | |
| Appetite suppression | − | | ++ | | |
| Anxiolytic | | | | | GW data |
| Cardiovascular Effects | | | | | |
| Bradycardia | − | | + | | Smiley et al, |
| | | | | | 1976 |
| Tachycardia | + | | − | | |
| Hypertension § | + | | − | | |
| Hypotension § | − | | + | | Adams et al, |
| | | | | | 1977 |
| Anti-inflammatory | ± | | ± | | Brown, 1998 |
| Immunomodulatory/ | | | | | |
| anti-inflammatory | | | | | |
| activity | | | | | |
| Raw Paw Oedema | − | | ++ | | GW data |
| Test | | | | | |
| Cox 1 | | | | | GW data |
| Cox 2 | | | | | GW data |
| TNFá Antagonism | + | + | ++ | ++ | |
| Glaucoma | ++ | | + | | |

*Effect is CB1 receptor independent.
† THC is pro convulsant
§ THC has a biphasic effect on blood pressure; in naïve patients it may produce postural hypotension and it has also been reported to produce hypertension on prolonged usage. GW Internal Report No 002/000159.

From these pharmacological characteristics and from direct experiments carried out by the applicant it has been shown, surprisingly, that combinations of THC and CBD in varying proportions are particularly useful in the treatment of certain therapeutic conditions. It has further been found clinically that the toxicity of a mixture of THC and CBD is less than that of THC alone.

Accordingly, in a further aspect the present invention provides compositions according to the invention which comprise specific ratios of CBD to THC, and which are clinically useful in the treatment or management of specific diseases or medical conditions.

In a further aspect the invention also provides compositions which have specific ratios of tetrahydrocannabinovarin (THCV) or cannabidivarin (CBDV). THCV and CBDV (propyl analogues of THC and CBD, respectively) are known cannabinoids which are predominantly expressed in particular Cannabis plant varieties and it has been found that THCV has qualitative advantageous properties compared with THC and CBD respectively. Subjects taking THCV report that the mood enhancement produced by THCV is less disturbing than that produced by THC. It also produces a less severe hangover.

In a still further aspect the invention provides compositions which have specific ratios of THCV to THC. Such compositions have been found to be particularly useful in the field of pain relief and appetite stimulation.

The invention also provides for administration of the above-described compositions containing specific ratios of cannabinoids in the form of a therapeutic vapour (or condensate) using the method of the invention.

The invention also provides methods of making a therapeutic vapour by heating of the aforementioned compositions under defined conditions, as well as methods of using the vapour so-produced to treat or manage specific diseases or conditions.

It has particularly been observed by the present applicants that the combinations of the specific cannabinoids are more beneficial than any one of the individual cannabinoids alone. Preferred embodiments are those compositions in which the amount of CBD is in a greater amount by weight than the amount of THC. Such compositions are designated as “reverse-ratio” compositions and are novel and unusual since, in the various varieties of medicinal and recreational Cannabis plant available world-wide, CBD is the minor cannabinoid component compared to THC. In other embodiments THC and CBD or THCV and CBDV are present in approximately equal amounts or THC or THCV are the major component and may be up to 95.5% of the total cannabinoids present.

Particularly preferred embodiments and the target medical conditions for which they are suitable are shown in Table 3 below.

TABLE 3
Target Therapeutic Groups for Different
Ratios of Cannabinoid

| Product group | Ratio THC:CBD | Target Therapeutic Area |
|---|---|---|
| High THC | >95:5 | Cancer pain, migraine, |
| | | appetite |
| | | stimulation |
| Even ratio | 50:50 | Multiple sclerosis, spinal |
| | | cord injury, peripheral |
| | | neuropathy, other |
| | | neurogenic pain. |
| Reverse/Broad | <25:75 | Rheumatoid arthritis, |
| ratio CBD | | Inflammatory bowel |
| | | diseases. |
| High CBD | <5:95 | Psychotic disorders |
| | | (schizophrenia), |
| | | Epilepsy & movement disorders |
| | | Stroke, head injury, |
| | | Disease modification in RA |
| | | and other inflammatory |
| | | conditions |
| | | Appetite suppression |

A principal advantage of the method of the invention is the ability to administer therapeutic substances in the form of a vapour which is substantially free of the products of pyrolysis, and in particular which is substantially free of the products of pyrolysis of vegetable matter.

Pyrolysis of vegetable matter generally occurs at about 218° C. Thus, one would assume that the maximum temperature of operation would be 218° C. This is not the case with the method of the present invention, however. The inventors have determined that because the heating time is short (the time period envisaged is less than 10 sec, preferably 0.1 to 5 sec, and most preferably about 1 sec), higher temperatures can be reached enabling compositions to be volatilised safely, without products of pyrolysis being produced, at significantly higher temperatures than predicted. This opens the way for administration of many more therapeutically active substances than one might have otherwise envisaged. It also offers the opportunity to include in the compositions therapeutic substances which have a boiling point higher than 218° C., so long as they have a boiling point or produce substantial vapour pressure at a temperature below 500° C., and more preferably below 375° C. Surprisingly, it has been found that it is possible to generate vapour from substances which have a higher boiling point than 218° C., but which have appreciable vapour pressure at temperatures in the range 130-195° C.

By selective admixture of a therapeutic substance with one or more additional carrier substances it is possible to reduce the temperature at which vaporisation occurs, i.e. to reduce the “effective boiling point” of the therapeutic substance, such that vaporisation occurs at a temperature below the boiling point of the therapeutic substance, and also to improve the efficiency of vapour generation.

In a preferred embodiment the compositions according to the invention may comprise one or more inert, non-combustible carriers or solvents, in addition to the therapeutic substances.

Preferred inert, non-combustible carrier substances include diatomaceous earth compounds, clays, silicates, carbonates, sulphites or sulphates of mono-dibasic metals or a mixtures thereof. Bentonite is a preferred example. Preferred solvents include ethanol, as it will evaporate off.

In a preferred embodiment the compositions may further comprise one or more hydrated salts which on heating release water of crystallisation and thereby modify the humidity and temperature of the vapour produced from the composition.

Preferred hydrated salts are pharmaceutically acceptable salts of metals in group 1 or 2 of the Periodic table which are solids, but yield water of crystallisation when heated. This release of water of crystallisation has the effect of extracting latent heat and thereby reducing the temperature of vaporisation. In addition, release of water of crystallisation humidifies the vapour produced by heating the composition and thereby improves patient acceptability.

A further advantageous feature of the invention is the possibility to include in the compositions non-volatile (at room temperature) pharmacologically inactive precursors of therapeutically substances which can be converted into pharmacologically active, volatile forms by heating or by a change of pH.

By way of example, cannabinoids may be included in the composition in the inactive acid form. The principal active constituents of cannabis plants, particularly Cannabis saliva and Cannabis indica, are the cannabinoids tetrahydrocannabinol (THC) and cannabidiol (CBD). Other cannabinoids such as cannabigerol (CBG), cannabichromene (CHC) and other cannabinoids are present in small quantities in harvested cannabis plants. The majority of cannabinoids are present in the plant as the corresponding carboxylic acids. The carboxylic acids themselves have little or no biological activity and in the production of cannabinoids for medicinal use it is necessary to convert the cannabinoid acids into free cannabinoids. Thus, when preparing extracts of cannabis for medicinal use by extraction with ethanol or supercritical CO₂ it is usual to preheat the cannabis in order to decarboxylate the cannabinoid acids to free cannabinoids.

With the present invention a separate decarboxylation step is not necessary at any stage of the preparation of the medicinal cannabis extract, since the cannabinoid acids present in a cannabis extract may be decarboxylated to give the active free cannabinoid form and simultaneously vaporised by the application of heat in the vaporisation step. This has implications for the delivery of active cannabinoids by inhalation, since it is possible to formulate a composition for delivery of cannabinoids as a vapour from an extract from a cannabis plant in which the majority of the cannabinoids are present in the inactive acid form without the need for de-carboxylation of the extract.

A suitable crude cannabis extract may be prepared by solvent extraction using a mixture of alcohol and water. The use of such mixtures reduces the lipophilicity of the solvent system and leads to proportionately greater extraction of cannabinoid acids. The extraction of cannabinoid acids in progressively more dilute alcohols is increased at high pH. The solvent extract may be prepared using conventional techniques known in the art such as, for example, maceration, percolation and reflux (Soxhlet) extraction.

In a further embodiment of the invention, compositions for delivery as a vapour may be formed by admixture of salts of esters of alkaloids with alkali or alkaline salts. The salts and esters of alkaloids are relatively non-volatile but when admixed with an alkali or alkaline salt are converted into the free alkaloid which is volatile. Pure compounds which are non-volatile in the salt form may also be converted into volatile substances by the application of heat.

In the methods of the invention the composition is heated to a defined temperature for a defined period of time, thereby producing a vapour comprising the therapeutic substances, or precursors thereof. The step of heating the composition may be carried out using any means known in the art which are suitable for this purpose. A preferred method of vaporisation involves placing the composition on an inert matrix or support which is then heated.

In a preferred embodiment, vaporisation of the composition may be carried out using a vaporiser apparatus which is the subject of a parallel application. The device consists of a heater which provides energy to vaporise the composition. The volume of vapour so produced is less than the mean respiratory tidal volume of air of a human subject and this charge of vapour is then admixed with inspired air during the act of breathing in. The compositions provide unit dose formulations which are intended for producing sufficient vapour to be taken in during one to several breaths. The device contains a resistive element on which the composition is deposited and a source of electrical power which is applied to heat the resistive element and thereby vaporise components of the composition. The device may further include a system of one-way valves which allow efficient inspiration of the vapour-laden air.

The invention will be further understood with reference to the following non-limiting experimental examples.

### EXAMPLE 1

### Preparation of a Cannabis Based Medicine Extract

2.5 Kg of dried cannabis biomass is reduced to a coarse powder and packed into the chamber of a Supercritical Fluid Extractor and extracted for 8 hours at a temperature of 40° C. at a pressure of 400 bar. The resulting extract is dissolved in 2 parts (by weight) of ethanol BP, chilled to −20° C., the precipitated plant ballast removed by filtration and evaporated to remove solvent. This extract is referred to as Cannabis Based Medicine Extract (CBME) and is the Botanical Drug Substance, used in the preparation of products in some of the following examples.

### EXAMPLE 2

### Composition for Administration of Cannabinoids by Inhalation

| | |
|---|---|
| High THC extract of cannabis | 10 parts |
| High CBD extract of cannabis | 10 parts |
| Bentonite | 2 parts |

Ethanol 90% BP a sufficient quantity (qs) to produce a pourable suspension.

Portions of the suspensions are applied to an inert matrix, and allowed to dry, forming a dose unit. When the dose unit is heated at a temperature between 130° C. and 225° C. (preferably 160-180° C.) the cannabinoid is vaporised, and can be inhaled by the patient.

The suspension can be applied as discreet drops to the matrix or by a screen-printing technique to cover the area of an electrical resistence which is used as the heating element. The solvent is allowed to evaporate off at room temperature.

The proportions of cannabis extract give an approximately 50/50 mix of THC and CBD.

### EXAMPLE 3

### Composition for Administration of Cannabinoids by Inhalation

| | |
|---|---|
| High THC extract of cannabis | 20 parts |
| Sodium sulphate decahydrate | 5 parts |
| (finely powdered) | |
| Sodium Sulphite | 0.5 parts |
| Bentonite | 2 parts |

Ethanol qs to produce a suspension.

The composition produced by this procedure is an example of the composition which has a high ratio of THC to CBD. It also contains sodium sulphite as hydrated salts. When heated, the hydrated salts yield up their water of crystallisation. The vaporisation of water withdraws heat and serves to reduce the maximum temperature achieved, allowing a higher initial rate of heating to be used to vaporise the medicament. The presence of water vapour also augments the volatilisation of cannabinoids and other constituents in the extract. Sodium sulphite acts as a chemical antioxidant during storage, and during volatilization. The amount of sulphur dioxide liberated is below that at which irritation of the respiratory tract occurs.

### EXAMPLE 4

### Composition for Administration of Cannabinoids by Inhalation

| | |
|---|---|
| High CBD cannabis extract | 20 parts |
| Calcium sulphate (dihydrate) | 5 parts |
| Sodium carbonate decahydrate | 5 parts |

Ethanol—sufficient quantity to produce a suspension.

The composition, when applied to an inert matrix as a thin layer or discrete drop, dries to give a dosage form which on heating, yields a vapour in which the ratio of CBD/THC is 30:1.

The examples given above are illustrative, and persons skilled in the art will appreciate that it is possible by varying the quantities of ingredients to achieve intermediate ratios of cannabinoid which may be appropriate for treatment of specific therapeutic conditions.

### EXAMPLE 5

### Composition for Administration of Ephedrine by Inhalation

| | | |
|---|---|---|
| Ephedrine sulphate | 20 | parts |
| Povidone | 1 | part |
| Bentonite | 2 | parts |
| Sodium phosphate (tribasic) | 0.5 | parts |
| dodecahydrate | | |

Alcohol, sufficient to produce a pourable suspension.

This formulation when applied drop-wise or as a uniform film to the surface of the substrate provides a dosage form which, when heated, produces a vapour of ephedrine. The tribasic sodium phosphate yields water of crystallisation which facilitates the generation of vapour. Until heated the products remains dry, and when heated the basic phosphate liberates ephedrine alkaloid from the stable form—ephedrine hydrochloride. Sufficient of the composition is present in the dosage form to give a quantity of 5-20 mg of ephedrine, by inhalation, suitable for the treatment of asthma and other conditions requiring bronchodilation.

### EXAMPLE 6

### Growing of Medicinal Cannabis

Plants are grown as clones from germinated seed, under glass at a temperature of 25° C.±1.5° C. for 3 weeks in 24 hour daylight; this keeps the plants in a vegetative state. Flowering is induced by exposure to 12 hour day length for 8-9 weeks.

No artificial pesticides, herbicides, insecticides or fumigants are used. Plants are grown organically, with biological control of insect pests.

The essential steps in production from seed accession to dried Medicinal Cannabis are summarised as follows:

Seed Accessions
↓
Seeds germinated at G-Pharm (UK)
↓
Selection for cannabinoid content and vigour
↓
Mother Plant
↓
Cuttings rooted
14-21 days in peat plug
25 C., 24 hour day length
↓
Rooted cuttings potted up in 5 liter pots of bespoke compost
↓
Young Clone Plant established
3 weeks, 24 hour day length, 25° C.
↓
Lower Branches Removed end of week 3
Used to make new generation of cuttings
↓
Induction of flowering
Plant relocation to 12 hour day length are to induce flowering
↓
Flower formation and maturation
8-9 weeks at 25° C.
↓
Harvest
90% of flowers and leaves senesced
↓
Drying
Under conditions of light exclusion
↓
MEDICINAL CANNABIS

### EXAMPLE 7

### Preparation of Herbal Drug Extracts

A flow chart showing a process which can be used for manufacture of extracts from High-THC and High-CBD cannabis chemovars is given below:

Medicinal Cannabis (High-THC or High-CBD)
↓
Chopping to predominantly 2 to 3 mm
↓
Heating at 100 to 150° C. for sufficient time to
decarboxylate acid form of
cannabinoids to produce neutral cannabinoids
↓
Extraction with a specified volume of liquid carbon
dioxide over 6 to 8 hours
↓
Removal of CO₂ by depressurisation
to recover crude extract
↓
“Winterisation”-Dissolution of crude extract in
ethanol Ph. Eur. followed by chilling solution
(−20° C./48 hrs) to precipitate unwanted waxes
↓
Removal of unwanted waxy material by cold filtration
↓
Removal of ethanol from the filtrate by
thin film evaporation under reduced pressure

The step of heating at 100 to 150° C. for sufficient time to decarboxylate acid form of cannabinoids to produce neutral cannabinoids may be omitted, since cannabis medicinal extracts wherein the majority of cannabinoids are present in the inactive acid form may be administered directly as a vapour using the method of the invention. Decarboxylation and vaporisation to produce a therapeutic vapour comprising the free cannabinoids may be accomplished in a single vaporisation step.

### EXAMPLE 8

High THC cannabis was grown under glass at a mean temperature of 21+2° C., RH 50-60%. Herb was harvested and dried at ambient room temperature at a RH of 40-45% in the dark. When dry, the leaf and flower head were stripped from stem and this dried biomass is referred to as “medicinal cannabis”.

Medicinal cannabis was reduced to a coarse powder (particles passing through a 3 mm mesh) and packed into the chamber of a Supercritical Fluid Extractor. Packing density was 0.3 and liquid carbon dioxide at a pressure of 600 bar was passed through the mass at a temperature of 35° C. Supercritical extraction is carried out for 4 hours and the extract was recovered by stepwise decompression into a collection vessel. The resulting green-brown oily resinous extract is further purified. When dissolved in ethanol BP (2 parts) and subjected to a temperature of −20° C. for 24 hours a deposit (consisting of fat-soluble, waxy material) was thrown out of solution and was removed by filtration. Solvent was removed at low pressure in a rotary evaporator. The resulting extract is a soft extract which contains approximately 60% THC and approximately 6% of other cannabinoids of which 1-2% is cannabidiol and the remainder is minor cannabinoids including cannabinol. Quantitative yield was 9% w/w based on weight of dry medicinal cannabis.

A high CBD chemovar was similarly treated and yielded an extract containing approximately 60% CBD with up to 4% tetrahydrocannabinol, within a total of other cannabinoids of 6%.

A person skilled in the art will appreciate that other combinations of temperature and pressure (in the range +10° C. to 35° C. and 60-600 bar) can be used to prepare extracts under supercritical and subcritical conditions.

1. A method of making a medicament which is a vapour comprising or consisting of at least one therapeutic substance or a precursor thereof, which method comprises heating a composition to a temperature not exceeding 500° C. for a time not exceeding 10 seconds and thereby generating a vapour comprising or consisting of at least one therapeutic substance or a precursor thereof, wherein the composition is non-volatile at 25° C. but is capable of generating a vapour comprising at least one therapeutic substance or a precursor thereof which is substantially free of any products of pyrolysis when heated to a temperature not exceeding 500° C. for a time not exceeding 10 seconds.

2. A method of administering a vapour or its condensate comprising or consisting of at least one therapeutic substance or a precursor thereof by inhalation, which method comprises
heating a composition to a temperature not exceeding 500° C. for a time not exceeding 10 seconds to generate a vapour comprising or consisting of at least one therapeutic substance or a precursor thereof in a portion of air smaller than the mean respiratory tidal volume, and
inhaling the vapour so-produced or its condensate in admixture with inspired air, wherein the composition is non-volatile at 25° C. but is capable of generating a vapour comprising at least one therapeutic substance or a precursor thereof which is substantially free of any products of pyrolysis when heated to a temperature not exceeding 500° C. for a time not exceeding 10 seconds.

3. A method according to claim 1 or claim 2 wherein the composition is heated to a temperature in the range 100-500° C., more preferably 100-400° C., more preferably 100-300° C., more preferably 150-250° C., and wherein the composition is capable of generating a vapour which is substantially free of any products of pyrolysis when heated to this temperature.

4. A method according to claim 1 or claim 2 wherein the composition is heated for a period of time in the range 0.1-5 seconds, and most preferably about 1 second, and wherein the composition is capable of generating a vapour which is substantially free of any products of pyrolysis when heated for this period of time.

5. A method according to claim 1 or claim 2 wherein the therapeutic substance (s) generated from the composition have a boiling point or produce substantial vapour pressure in the range 75° C.-500° C., more preferably 180° C.-375° C.

6. A method according to claim 1 or claim 2 wherein the composition comprises at least one therapeutic substance or a precursor thereof.

7. A method according to claim 6 wherein the composition comprises at least one precursor of a therapeutic substance which is converted from a pharmacologically inactive form into a pharmacologically active form by the application of heat.

8. A method according to claim 6 wherein the therapeutic substance included in the composition is at least one cannabis extract.

9. A method according to claim 8 wherein the composition consists of at least one cannabis extract.

10. A method according to claim 8 wherein the cannabis extract is a solvent extract prepared by solvent extraction using a mixture of alcohol and water.

11. A method according to claim 8 wherein the cannabis extract has not been subject to any decarboxylation step to convert cannabinoid acids to free cannabinoids.

12. A method according to claim 6 wherein the therapeutic substance included in the composition comprises one or more natural or synthetic cannabinoids.

13. A method according to claim 12 wherein the therapeutic substance comprises tetrahydrocannabinol, Δ⁹-tetrahydrocannabinol, Δ⁹-tetrahydrocannabinol propyl analogue, cannabidiol, cannabidiol propyl analogue, cannabinol, cannabichromene, cannabichromene propyl analogue, cannabigerol or any mixture thereof.

14. A composition formulated for administration of a vapour or its condensate, which vapour comprises or consists of at least one therapeutic substance or a precursor thereof, wherein the composition is non-volatile at 25° C. but is capable of generating a vapour comprising at least one therapeutic substance or a precursor thereof which is substantially free of any products of pyrolysis when heated to a temperature not exceeding 500° C. for a time not exceeding 10 seconds.

15. A composition according to claim 14 wherein the composition is capable of generating a vapour which is substantially free of any products of pyrolysis when heated to a temperature in the range 100-500° C., more preferably 200-500° C., more preferably 300-500° C., more preferably 400-500° C.

16. A composition according to claim 14 wherein the composition is capable of generating a vapour which is substantially free of any products of pyrolysis when heated for a period of time in the range 0.1-5 seconds, and most preferably about 1 second.

17. A composition according to claim 14 wherein the therapeutic substance(s) generated from the composition have a boiling point or produce substantial vapour pressure in the range 75° C.-500° C., more preferably 180° C.-375° C.

18. A composition according to claim 14 which comprises at least one therapeutic substance or a precursor thereof.

19. A composition according to claim 18 which further includes at least one solvent or inert, non-combustible carrier.

20. A composition according to claim 19 which includes as a carrier a diatomaceous earth compound, a clay, a silicate, a carbonate, sulphite or sulphate of a mono-dibasic metal or a mixture thereof.

21. A composition according to claim 19 or claim 20 which includes ethanol as a solvent.

22. A composition according to claim 18 which further comprises a hydrated salt which on heating releases water of crystallisation and thereby modifies the humidity and temperature of the vapour produced from the composition.

23. A composition according to claim 22 wherein the hydrated salt is a pharmaceutically acceptable salt of a metal in group 1 or 2 of the Periodic table containing water of crystallisation.

24. A composition according to claim 18 which comprises at least one precursor of a therapeutic substance which is converted from a pharmacologically inactive form into a pharmacologically active form by the application of heat.

25. A composition according to claim 18 wherein the therapeutic substance is at least one cannabis extract.

26. A composition according to claim 25 wherein the cannabis extract is a solvent extract prepared by solvent extraction using a mixture of alcohol and water.

27. A method according to claim 25 wherein the cannabis extract has not been subject to any decarboxylation step to convert cannabinoid acids to free cannabinoids.

28. A composition according to claim 18 wherein the therapeutic substance comprises one or more natural or synthetic cannabinoids.

29. A composition according to claim 28 wherein the therapeutic substance comprises tetrahydrocannabinol, Δ⁹-tetrahydrocannabinol, Δ⁹-tetrahydrocannabinol propyl analogue, cannabidiol, cannabidiol propyl analogue, cannabinol, cannabichromene, cannabichromene propyl analogue, cannabigerol or any mixture thereof.

30. A vapour which is obtainable by heating a composition according to claim 14 to a temperature not exceeding 500° C. for a time not exceeding 10 seconds.
